(19) United States
(12) Patent Application Publication
(10) Pub. No.: US 2024/0409984 A1
(43) Pub. Date: Dec. 12, 2024

(54) METHODS FOR DETECTING MUTATIONS IN TARGET NUCLEIC ACIDS USING DUAL PROBES

(71) Applicant: REVVITY HEALTH SCIENCES, INC., Waltham, MA (US)

(72) Inventors: Zhixiang Lu, Belmon, MA (US); Nidhi Nandu, Arlington, MA (US); Yanhong Tong, Boxford, MA (US)

(21) Appl. No.: 18/208,140

(22) Filed: Jun. 9, 2023

(51) Int. Cl. C12Q 1/6827; C12Q 1/689

(52) U.S. Cl. CPC C12Q 1/6827; C12Q 1/689; C12Q 2600/156; C12Q 2600/166

(57) ABSTRACT

Provided herein are dual probe assays for detecting a mutation in an amplified target nucleic acid, e.g., for detecting a single point mutation linked to drug resistance.

### BACKGROUND

Mutations in nucleic acids can impact a wide range of important biological processes including disease development, disease susceptibility, and drug efficacy. Mutations can be detected using PCR-based assays, which in turn can require instrumentation not compatible with resource-limited settings. As an alternative method to PCR-based assays, isothermal amplification-based assays can be used rapidly amplify the nucleic acids at a constant temperature while eliminating the need for thermal cycling equipment.

### SUMMARY

The present disclosure is based, at least in part, on the development of accurate and sensitive assays for detection of a mutation in an amplified target nucleic acid. In some examples, a plurality of probes can be used, in which at least one probe is a calibrator probe (e.g., any described herein). The experimental data provided herein demonstrated that such assays can be used to detect a single point mutation in Mycobacterium tuberculosis DNA that is linked to rifampicin resistance. Other mutations from other sources can be detected (e.g., by use of probe sequences for such mutations).

Accordingly, aspects of the present disclosure provide a method for detecting a mutation in an amplified target nucleic acid in a sample, the method comprising incubating a test sample comprising an amplified target nucleic acid; a calibrator probe comprising a first nucleic acid sequence that is sufficiently complementary to a second nucleic acid sequence of the amplified target nucleic acid, wherein the second nucleic acid sequence of the amplified target nucleic acid comprises a wild-type sequence, and wherein the calibrator probe comprises a first detectable label; an indicator probe comprising a third nucleic acid sequence that is sufficiently complementary to a fourth nucleic acid sequence of the amplified target nucleic acid, wherein the fourth nucleic acid sequence of the amplified target nucleic acid comprises an absence or a presence of a mutation, and wherein the indicator probe comprises a second detectable label; wherein the test sample is incubated under conditions sufficient for hybridizing the calibrator probe and/or the indicator probe to the amplified target nucleic acid; detecting a signal from the first detectable label and a signal from the second detectable label; and determining the absence or the presence of the mutation in the fourth nucleic acid sequence of the amplified target nucleic acid based on a score value determined from the signal from the first detectable label and the signal from second detectable label, wherein a deviation of the score value from a control value indicates the presence of the mutation in the fourth nucleic acid sequence of the amplified target nucleic acid in the test sample.

In some embodiments, the score value comprises a Z-score of

$$Z = \vert \frac{X - μ}{σ} \vert ,$$

X comprises a signal from the test sample based on the signal from the first detectable label and the signal from second detectable label, μ comprises a signal from a plurality of negative control samples comprising an amplified target nucleic acid without a mutation, and σ comprises a standard deviation of the signal from the plurality of negative control samples comprising the amplified target nucleic acid without the mutation.

In some embodiments, the control value is based on an absolute value determined from the signal from the first detectable label and the signal from the second detectable label in a negative control sample comprising the amplified target nucleic acid without the mutation and/or based on an absolute value determined from the signal from the first detectable label and the signal from the second detectable label in a positive control sample comprising the amplified target nucleic acid with the mutation. In some embodiments, the control value is a predetermined threshold value.

In some embodiments, determining the absence or the presence of the mutation comprises assessing a presence or an absence of a calibrator negative signal or an indicator negative signal; calculating a Z-score of

$$Z = \vert \frac{X - μ}{σ} \vert ,$$

wherein X comprises a signal from the test sample based on the signal from the first detectable label and the signal from second detectable label, μ comprises a signal from a plurality of negative control samples comprising an amplified target nucleic acid without a mutation, and σ comprises a standard deviation of the signal from the plurality of negative control samples comprising the amplified target nucleic acid without the mutation; comparing the Z-score to a threshold value of T; and determining the presence of the mutation in the fourth nucleic acid sequence of the amplified target nucleic acid when the Z-score is greater than the threshold value (e.g., Z>T) or determining the absence of the mutation in the fourth nucleic acid sequence of the amplified target nucleic acid when the Z-score is less than or equal to the threshold value (e.g., Z≤T).

In some embodiments, the mutation is an insertion, a deletion, or a substitution. In some embodiments, the mutation is a single nucleotide polymorphism.

In some embodiments, the amplified target nucleic acid comprises genomic DNA. In some embodiments, the amplified target nucleic acid is a viral nucleic acid, a bacterial nucleic acid, a fungal nucleic acid or a mammalian nucleic acid. In some embodiments, the amplified target nucleic acid is a nucleic acid from Mycobacterium tuberculosis. In some embodiments, the amplified target nucleic acid comprises a rifampicin resistance-determining region (RRDR) from Mycobacterium tuberculosis. In some embodiments, the RRDR comprises a mutation in a codon selected from codon 511, codon 516, codon 526, codon 531, codon 533, codon 577, or a combination thereof.

In some embodiments, the calibrator probe has a length of 20 to 50 nucleic acids and/or the indicator probe has a length of 20 to 50 nucleic acids. In some embodiments, the calibrator probe is completely complementary to the second nucleic acid sequence of the amplified target nucleic acid and/or the indicator probe is completely complementary to the fourth nucleic acid sequence of the amplified target nucleic acid.

In some embodiments, the calibrator probe comprises at least one modification and/or wherein the indicator probe comprises at least one modification. In some embodiments, the at least one modification is internally located in the calibrator probe and/or the indicator probe. In some embodiments, the at least one modification of the indicator probe is located within 5 to 10 nucleotides (e.g., downstream or upstream) of the mutation in the fourth nucleic acid sequence of the amplified target nucleic acid when the indicator probe is hybridized to the fourth nucleic acid sequence. In some embodiments, the at least one modification comprises locked nucleic acid (LNA), a peptide nucleic acid (PNA), a bridged nucleic acid (BNA), an unlocked nucleic acid (UNA), and/or a self-avoiding molecular recognition system (SAMRS).

In some embodiments, the first and second detectable labels are selected from the group consisting of a fluorescent label, a radioactive label, a colorimetric, a chemiluminescent label, and/or a dye.

In some embodiments, the first detectable label comprises a first fluorophore and a first quencher, and wherein the second detectable label comprises a second fluorophore and a second quencher. In some embodiments, the first quencher and the second quencher are the same quencher. In some embodiments, the first fluorophore and the second fluorophore are selected from the group consisting of FAM, TAMRA, HEX, ROX, VIC, Cy™ 3, Cy™ 5, and Texas Red®. In some embodiments, the first quencher and the second quencher are selected from the group consisting of black hole quencher (BHQ), deep dark quencher (DDQ), Eclipse® quencher, Dabcyl, QSY® quencher, Iowa Black FQ, Iowa Black RQ, ZEN™ Quencher, and TAMRA.

In some embodiments, the calibrator probe comprises CGCGAGCCGGATGTTGATCAACGTCTGCTCGCG (SEQ ID NO:1). In some embodiments, the calibrator probe is X-CGCGAGCCGGATGTTGATCAACGTCTGCTCGCG-Y (SEQ ID NO:1), and wherein at least one X and Y is a fluorophore and at least one of X and Y is a quencher.

In some embodiments, the indicator probe comprises CGCGAGACCCACAAGCGCCGACTGTCGGCGCTCGCG (SEQ ID NO:2). In some embodiments, the indicator probe is X-CGCGAGACCCACAAGCGCCGACTGTCGGCGCTCGCG-Y (SEQ ID NO:2), and wherein at least one X and Y is a fluorophore and at least one of X and Y is a quencher. In some embodiments, the indicator probe comprises at least one LNA modification at a position selected from positions 10, 11, 12, 25, 26, and 27 in SEQ ID NO:2. In some embodiments, the indicator probe comprises LNA modifications at positions 10, 11, 12, 25, 26, and 27 in SEQ ID NO:2.

In some embodiments, the calibrator probe and the indicator probe is a molecular beacon.

In some embodiments, the sample further comprises 3′-amino-2′,3′-dideoxyribonucleotide 5′-triphosphates (nNTPs), one or more additional primers, a buffer, and/or a DNA polymerase. In some embodiments, the one or more additional primers comprises another indicator probe comprising a fifth nucleic acid sequence that is complementary to a sixth nucleic acid sequence of the amplified target nucleic acid, wherein the sixth nucleic acid sequence of the amplified target nucleic acid comprises an absence or a presence of a mutation, and wherein the another indicator probe comprises a third detectable label.

In some embodiments, methods described herein further comprise (e.g., prior to providing the test sample): amplifying the amplified target nucleic acid using an isothermal amplification method. In some embodiments, the isothermal amplification method is selected from nucleic acid sequence based amplification (NASBA), helicase-dependent amplification (HDA), loop-mediated isothermal amplification (LAMP), strand displacement amplification (SDA), or recombinase polymerase amplification (RPA).

In some embodiments, detecting the signal from the first, the second, detectable label comprises real-time detection or end point detection. In some embodiments, detecting the signal from the first, the second detectable label comprises detection using a microfluidic device.

Aspects of the present disclosure provide a kit comprising: a calibrator probe comprising a first nucleic acid sequence that is sufficiently complementary to a second nucleic acid sequence of an amplified target nucleic acid, wherein the second nucleic acid sequence of the amplified target nucleic acid comprises a wild-type sequence, and wherein the calibrator probe comprises a first detectable label; an indicator probe comprising a third nucleic acid sequence that is sufficiently complementary to a fourth nucleic acid sequence of the amplified target nucleic acid, wherein the fourth nucleic acid sequence of the amplified target nucleic acid comprises an absence or a presence of a mutation, and wherein the indicator probe comprises a second detectable label; and instructions for performing a method of claims 1-36.

In some embodiments, the kit further comprises 3′-amino-2′,3′-dideoxyribonucleotide 5′-triphosphates (nNTPs), one or more additional primers, a buffer, and a DNA polymerase and/or a reverse transcriptase.

### DETAILED DESCRIPTION OF THE DRAWINGS

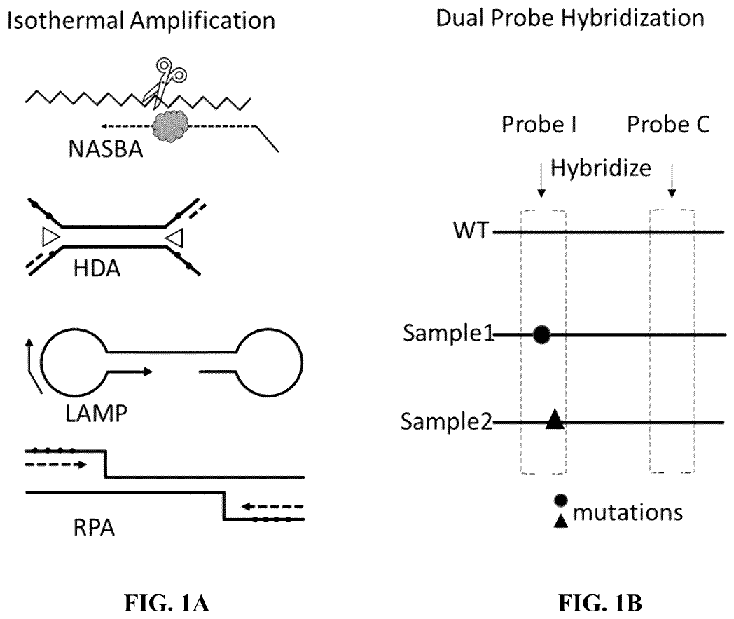

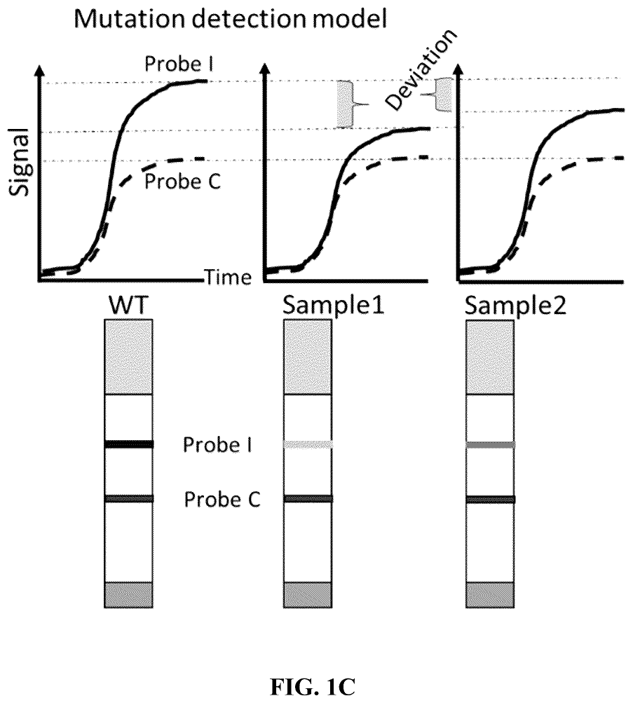

FIGS. 1A-1C are schematic diagrams illustrating a non-limiting dual probe method for detecting a mutation in an amplified target nucleic acid. FIG. 1A is a non-limiting schematic diagram showing various isothermal amplification methods that can be used to amplify a target nucleic acid. FIG. 1B is a non-limiting schematic diagram showing hybridization of dual probes to the amplified target nucleic acid. FIG. 1C is a non-limiting schematic diagram showing fluorescent amplification curves from wild type (WT) and test samples (Sample 1, Sample 2). Upper panel shows real-time detection. Lower panel shows endpoint detection. WT: Wild type; Probe I: “Indicator” probe; Probe C: “Calibrator” probe.

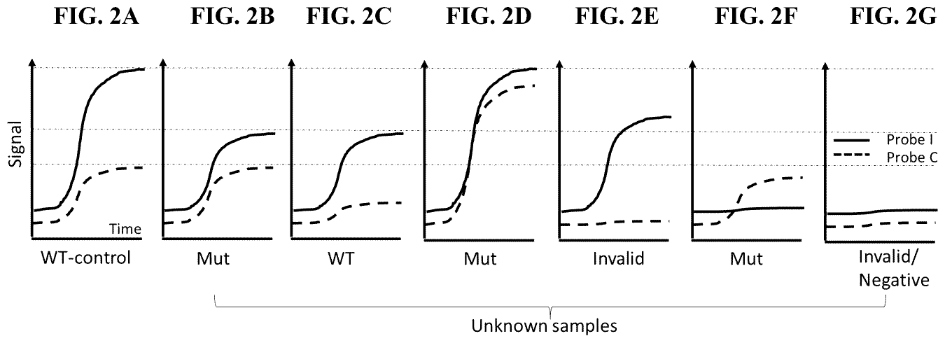

FIGS. 2A-2G are schematic diagrams illustrating non-limiting real-time amplification curves from detection of a mutation in an amplified target nucleic acid using dual probes. FIG. 2A: WT control sample. FIG. 2B, FIG. 2D, and FIG. 2F: Mutant test samples. FIG. 2C: WT test sample. FIG. 2E: Invalid test sample. FIG. 2G: Invalid/negative test sample.

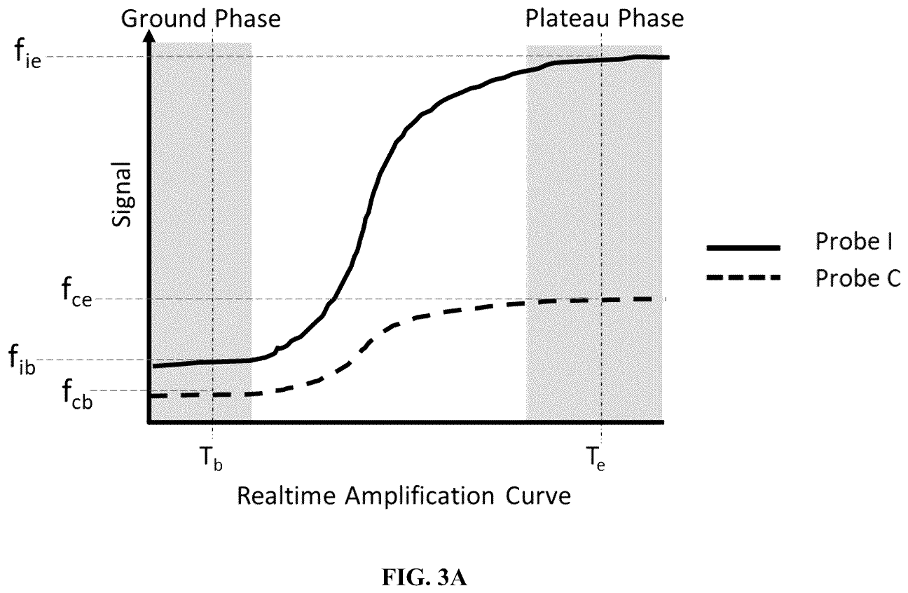

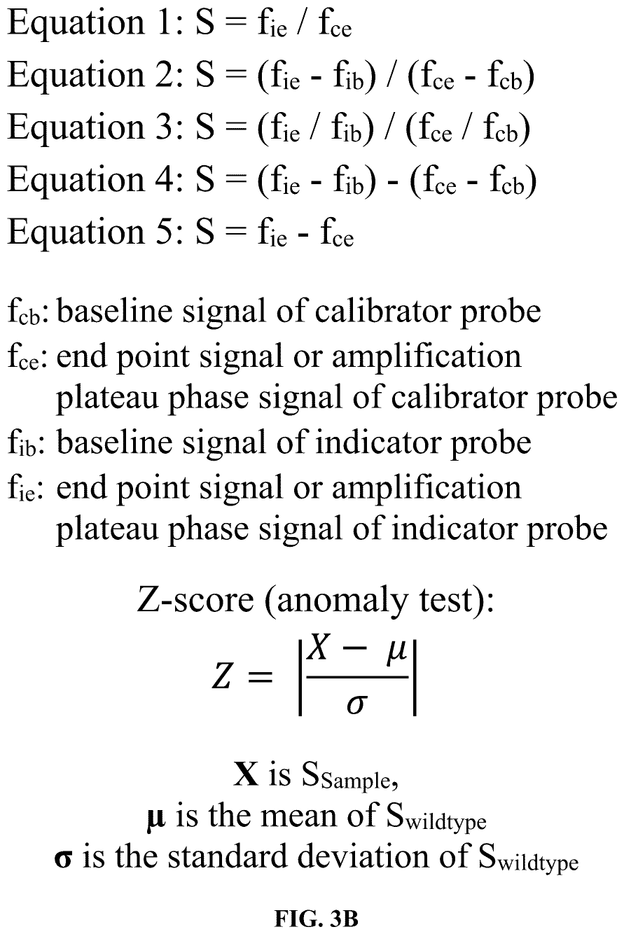

FIGS. 3A-3B are a schematic diagram illustrating a non-limiting real-time amplification calibration curve (FIG. 3A) and non-limiting equations for signal calibration (FIG. 3B). Z-score can be used to determine a presence or an absence of a mutation (FIG. 3B).

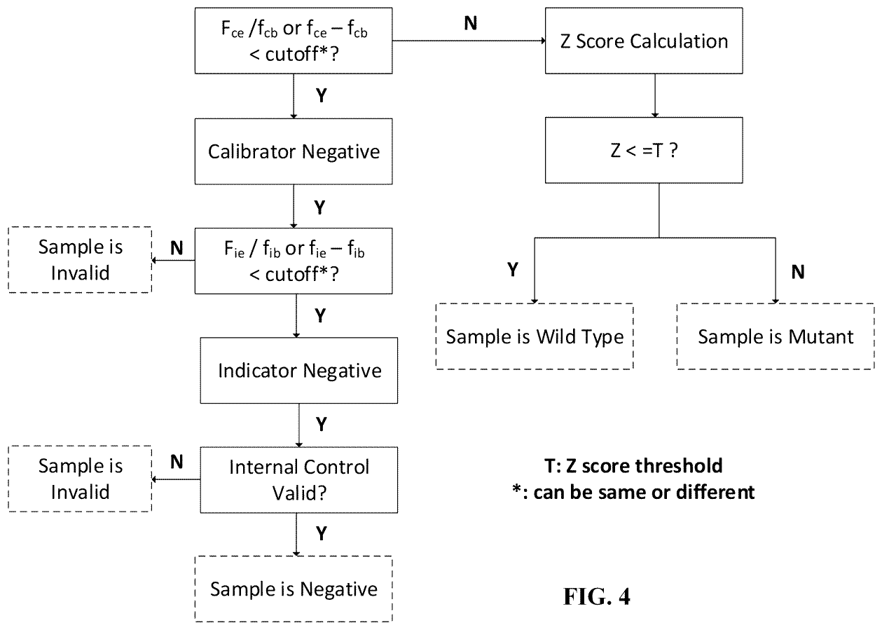

FIG. 4 is a flowchart showing a non-limiting analysis for determining whether a sample is a WT sample, a mutant sample, an invalid sample, or a negative sample.

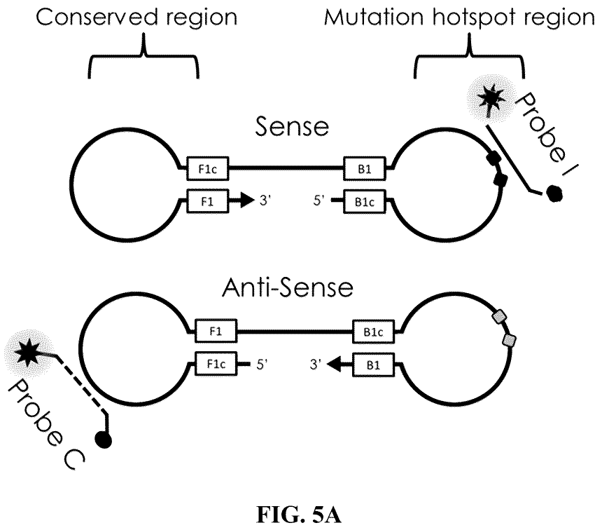

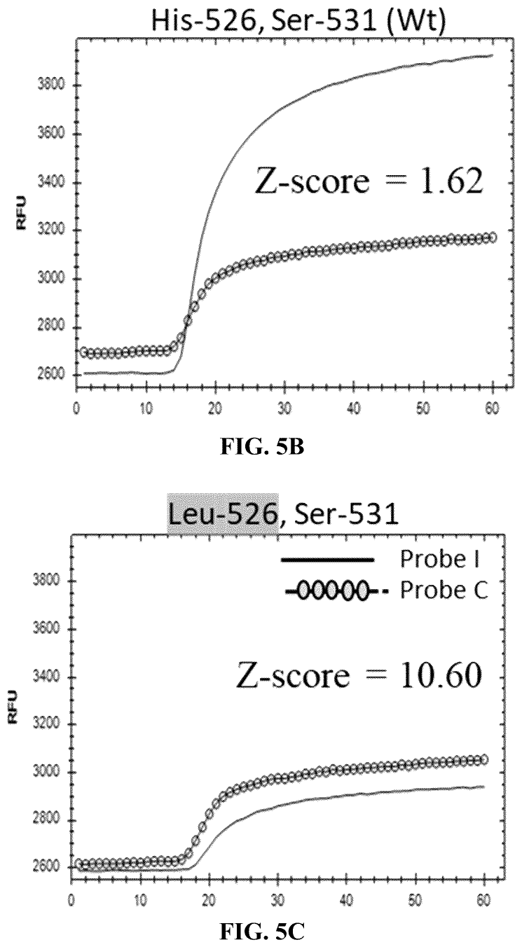

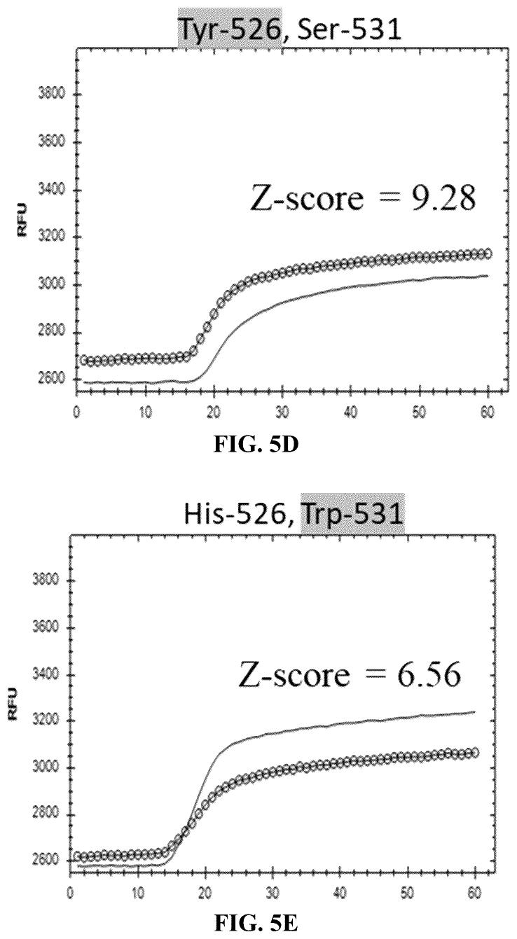

FIGS. 5A-5E are a schematic diagram illustrating a non-limiting probe design for detecting a mutation using loop-mediated isothermal amplification (LAMP) (FIG. 5A) and real-time amplification curves for various samples (FIGS. 5B-5E). FIG. 5B: Wild type plasmid (His-526, Ser-531). FIG. 5C: RIF 1 plasmid (Leu-526, Ser-531). FIG. 5D: RIF 2 plasmid (Tyr-526, Ser-531). FIG. 5E: RIF 3 plasmid (His-526, Trp-531). X-axis is time (min), Y-axis is fluorescence signal (RFU). Z-score is labeled in each sample's real-time amplification curve.

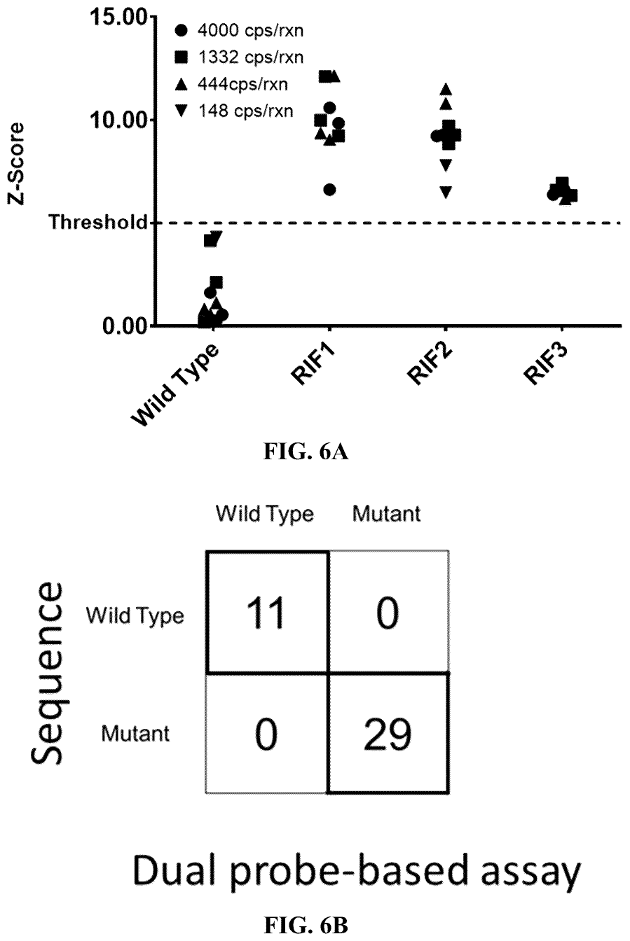

FIGS. 6A-6B are a non-limiting scatter plot of Z-scores of 40 samples (FIG. 6A) and a non-limiting plot showing accuracy of mutation detection using dual probes (FIG. 6B). In FIG. 6A, the initial sample concentration is labeled (as indicated by the legend using various symbols). The dashed line shows a Z-score=5 as the threshold.

### DETAILED DESCRIPTION

Provided herein are methods for detecting a mutation in a target nucleic acid (e.g., an amplified target nucleic acid) using a plurality of probes (e.g., dual probes), which include at least a calibrator probe and an indicator probe. Such methods involve hybridizing the calibrator probe and the indicator probe to the target nucleic acid, and determining whether a mutation is present based on whether a signal from the test sample deviates from a signal from the wild type sample. In some instances, methods described herein further comprise amplifying a target nucleic acid to yield an amplified target nucleic acid and hybridizing probes (e.g., a calibrator probe and an indicator probe) to the amplified target nucleic acid.

FIGS. 1A-1C provide a non-limiting schematic depiction of dual probe-based mutation detection using methods described herein. As shown in FIG. 1A, the target nucleic acid can be amplified using an isothermal amplification technique including, but not limited to, nucleic acid sequence based amplification (NASBA), helicase-dependent amplification (HDA), loop-mediated isothermal amplification (LAMP), and recombinase polymerase amplification (RPA). Other amplification methods can be employed, such as strand displacement Amplification (SDA), multiple strand displacement amplification (MDA), or rolling-circle amplification (RCA).

As shown in FIG. 1B, the calibrator probe hybridizes to a first wild-type sequence in the amplified target nucleic acid present in a WT sample (WT) or in a test sample (Sample 1, Sample 2). Here, the calibrator probe can be designed to hybridize to a region that typically lacks a mutation (e.g., a region having a certain sequence that is present in a wild-type sequence and that is present even in a test sequence suspected of having one or more mutations in another location). In some non-limiting embodiments, the calibrator probe includes a sequence that hybridizes to a sequence (e.g., a wild-type sequence) in a conserved region. As used herein, the term “conserved region” or “conserved sequence” refers to a nucleic acid sequence in a region of a target nucleic acid that is the same or highly similar across different groups such as different species or different strains (e.g., drug resistant strains).

The indicator probe hybridizes to a second wild-type sequence in the amplified target nucleic acid in a WT sample (WT), or the indicator probe hybridizes to a mutant sequence in the amplified target nucleic acid in a test sample (Sample 1, Sample 2). For instance, the indicator probe can be designed to hybridize to a region that can include a mutation (e.g., a region having a certain sequence that is typically absent in a wild-type sequence and that may be present in a test sequence suspected of having one or more mutations). In some non-limiting embodiments, the indicator probe includes a sequence that hybridizes to a sequence (e.g., a wild-type sequence) in a mutated region. The mutated region can be characterized as having same or different mutations between two or more test samples (e.g., differing in sequence and/or location of the mutation). As used herein, the term “mutated region” or “mutated sequence” refers to a nucleic acid sequence in a region of a target nucleic acid that is not conserved and varies across different groups such as different species or different strains (e.g., drug resistant strains).

Calibrator probes and indicator probes can be used to distinguish between sequences having or lacking one or more mutations. As shown in FIG. 1C, a mutation can be present when a calibrated signal from the test sample (Sample 1, Sample 2) deviates from a calibrated signal from the WT sample (WT). The absolute signal can be related, in part, to the amount of amplified product and the extent of hybridization efficiency between the amplified target nucleic acid and the probe. In some embodiments, the indicator probe can be sensitive to any sequence changes (e.g., different sequence mutations) in the region of hybridization. Methods described herein can include detection of the amplified target nucleic acid using real-time detection (FIG. 1C, upper panel) and/or endpoint detection (FIG. 1C, lower panel).

FIGS. 2A-2G shows schematic diagrams of non-limiting, possible real-time detection results from dual probe-based mutation detection methods described herein. Amplification curves of a WT sample (FIG. 2A) and test samples (FIGS. 2B-2G) are shown. As described in more detail below, the amplification curve of the WT sample can be compared to the amplification curve of the test sample to determine whether a mutant sequence is present in an amplified target nucleic acid in the test sample. Such comparisons can include, for example and without limitation, the use of a calibrated score (e.g., as described herein).

FIGS. 2A-2B show a non-limiting example of detection of a mutant sequence in an amplified target nucleic acid in the test sample. Detection of the mutant sequence can be indicated by a decrease in the indicator probe signal in the test sample, as compared to the indicator probe signal in the WT sample. Furthermore, a substantially similar calibrator probe signal in the test sample and the WT sample would provide a reference, such that the decrease in indicator probe signal in the test sample can likely be attributed to the decrease in indicator probe hybridization to the mutant sequence in the amplified target nucleic acid.

FIG. 2A and FIG. 2C show a non-limiting example of detection of a wild-type sequence in an amplified target nucleic acid in the test sample. Detection of the wild-type sequence can be indicated by a synchronous decrease in the indicator probe signal and the calibrator probe signal in the test sample, as compared to the WT sample. Since the calibrated score does not change, the identification of the test sample as a wild-type sample is not necessarily affected by the synchronous decrease in signal. For example and without limitation, such synchronous decrease in signal can be indicative of lower target concentrations and/or reaction inhibition, which decreases both signals from both probes.

FIG. 2A and FIG. 2D show a non-limiting example of detection of a mutant sequence in the test sample. Detection of the mutant sequence can be indicated by an increase in the indicator probe signal and the calibrator probe signal in the test sample, as compared to the WT sample. Since the calibrated score changes, the test sample can be identified as having a mutant sequence.

FIG. 2A and FIG. 2E show a non-limiting example of an invalid test sample, which can be indicated by a lack of signal from the calibrator probe and a non-specific signal from the indicator probe. The non-specific indicator probe signal could result from cross-contamination or non-specific amplification.

FIG. 2A and FIG. 2F show a non-limiting example of detection of a mutant sequence in an amplified target nucleic acid in the test sample. Detection of the mutant sequence can be indicated by absence of an indicator probe signal and presence of a calibration probe signal. Presence of the calibrator probe signal indicates presence of the amplified target nucleic acid, but absence of the indicator probe signal indicates presence of the mutation in the amplified target nucleic acid.

FIG. 2A and FIG. 2G show a non-limiting example of an invalid or negative test sample, which can be indicated by a lack of signal from the calibrator probe and the indicator probe. Without wishing to be limited by mechanism, the “negative” result could result from expired reagents, sample interference, and/or a low concentration or absence of a target nucleic acid or an amplified target nucleic acid.

Accordingly, described herein are improved methods for rapid detection of a mutation using dual probes including a calibrator probe and an indicator probe. The dual probe methods described herein utilize probe hybridization that allows sequence-specific detection of an amplified target nucleic acid. For example and without limitation, this can be achieved by hybridization of the calibrator probe and indicator probe to the amplified target nucleic acid with minimized (e.g., little to no) hybridization of the probes to nonspecific amplification products, thereby reducing false positives.

The calibrator probe can be used as an endogenous reference. In some non-limiting embodiments, it can serve as a control for the integrity of the reagents and the presence of inhibitors in the samples. Use of the calibrator probe may simplify the molecular diagnostics design and/or may reduce the cost of associated with additional control reactions. The calibrator probe may also be used to normalize the signal from the indicator probe. For example and without limitation, the signal from the indicator probe can vary in different reactions or at different sample concentrations. Because the calibrator probe can be present in the same reaction and targets the same amplified target nucleic acid as the indicator probe, the calibrator probe can serve as a ruler or reference to normalize the indicator probe's signal. As such, the use of dual probes can reduce both false positives and false negatives or reduce the prevalence of such false responses. In some non-limiting examples, the indicator probe can be completely complementary or substantially complementary to a wild-type sequence in the amplified target nucleic acid. Without wishing to be limited by mechanism, use of complementary sequences may provide enhanced sensitivity to any changes (e.g., mutations) in its sequence (e.g., changes in the target sequence, as compared to the wild-type sequence). As shown in FIGS. 1B-1C, the indicator probe can hybridize to a sequence in the amplified target nucleic acid that can have different mutations.

### I. Components for Use in Dual Probe Assays

The dual probe methods described herein involve a calibrator probe and an indicator probe, each of which can be conjugated to a detectable label, and an amplified target nucleic acid.

### Calibrator Probes and Indicator Probes

A probe refers to an oligonucleotide that includes a nucleotide sequence capable of hybridizing or annealing to a target nucleic acid (e.g., an amplified target nucleic acid). The probe can hybridize or anneal at or near a specific region of interest (also referred to as a target sequence) in the amplified target nucleic acid.

A calibrator probe refers to a probe that hybridizes to a wild-type sequence in a target nucleic acid (e.g., an amplified target nucleic acid). An indicator probe refers to a probe that hybridizes to an amplified target nucleic acid at a sequence having an absence or a presence of a mutation. As such, the indicator probe can hybridize to a wild-type sequence or a mutant sequence in an amplified target nucleic acid. The hybridization efficiency of the indicator probe to the mutant sequence can be less than that of the indictor probe to the wild-type sequence, which can allow for differentiation of a mutant sequence and a wild-type sequence.

A probe can be any length suitable for hybridizing to an amplified target nucleic acid. The probe can be the same length as the amplified target nucleic acid or the probe can be shorter in length than the amplified target nucleic acid. In some embodiments, the probe has a length of about 20 to about 50 nucleotides, e.g., about 25 to about 50 nucleotides, about 30 to about 50 nucleotides, about 35 to about 50 nucleotides, about 40 to about 50 nucleotides, about 45 to about 50 nucleotides, about 20 to about 45 nucleotides, about 20 to about 40 nucleotides, about 20 to about 35 nucleotides, about 20 to about 30 nucleotides, or about 20 to about 25 nucleotides.

All or a portion of a calibrator probe and/or an indicator probe can be completely complementary or substantially complementary to an amplified target nucleic acid. Substantially complementary refers to nucleotide sequences that will hybridize with each other. For example, the probe (e.g., calibrator probe and/or indicator probe) and the amplified target nucleic acid can be at least 75%, at least 80%, at least 85%, at least 90%, at least 95%, at least 96%, at least 97%, at least 98%, at least 99%, or more complementary to each other over any useful region (e.g., over a region of 8, 9, 10, 11, 12, 13, 14, 15, 16, 17, 18, 19, 20, 21, 22, 23, 24, 25, 30, 35, 40, 45, 50, or more nucleotides). Stringent conditions can refer to conditions under which a nucleic acid having complementarity or substantial complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

All or a portion of a calibrator probe and/or an indicator probe can comprise a nucleic acid sequence from a drug resistant bacteria, e.g., Mycobacterium tuberculosis, Mycoplasma genitalium, and Candida auris. Those skilled in the art will recognize that a nucleic acid sequence from any number of drug resistant bacteria may be utilized. For example, when detecting a drug resistant strain of Mycobacterium tuberculosis, the indicator probe can comprise a nucleic acid sequence of a rifampicin resistance-determining region (RRDR) in a rpoB gene from Mycobacterium tuberculosis. In such instances, the calibrator probe can comprise a nucleic acid sequence outside of the RRDR. For example, the indicator probe can comprise CGCGAGACCCACAAGCGCCGACTGTCGGCGCTCGCG (SEQ ID NO:2), and the calibrator probe can comprise CGCGAGCCGGATGTTGATCAACGTCTGCTCGCG (SEQ ID NO:1). See Examples below.

A probe can include any sequence, e.g., a naturally occurring sequence or a modified sequence. The probe can be obtained from one or more sources, e.g., isolated from a biological sample or obtained from commercial sources.

A probe can include any type of hybridization probe known in the art or described herein. Non-limiting examples of probes for use in methods described herein include molecular beacon probes, toehold probes, padlock probes, and combinations thereof. Other non-limiting examples include probes having a metastable hairpin structure (e.g., comprising a loop and a stem region). A probe can be single stranded or double stranded, or a combination thereof (e.g., a DNA/RNA hybrid).

A probe can include DNA, RNA, or a combination thereof (e.g., a DNA/RNA hybrid). A probe can include at least one modified nucleotide. Any modified nucleotide known in the art can be included in probes for use in methods described herein. Non-limiting examples of a modified nucleotide include a locked nucleic acid (LNA), a peptide nucleic acid (PNA), a bridged nucleic acid (BNA), an unlocked nucleic acid (UNA), and a self-avoiding molecular recognition system (SAMRS).

The calibrator probe can include at least one modified nucleotide, the indicator probe can include at least one modified nucleotide, or both the calibrator probe and the indicator probe can include at least one modified nucleotide. In some embodiments, a plurality of modified nucleotides can be present in a probe. For example, when detecting a drug resistant strain of Mycobacterium tuberculosis, the indicator probe can comprise at least one modified nucleotide (e.g., at least one LNA modified nucleotide) at a position selected from positions 10, 11, 12, 25, 26, and 27 in SEQ ID NO:2. In some embodiments, the indicator probe can comprise a modified nucleotide (e.g., a LNA modified nucleotide) at positions 10, 11, 12, 25, 26, and 27 in SEQ ID NO:2.

The at least one modified nucleotide can be included anywhere in the probe, e.g., 3′ end of the probe, 5′ end of the probe, internally, or a combination thereof. In some embodiments, the probe can include a modified nucleotide that is located within 5 to 10 nucleotides (e.g., downstream or upstream) of a mutation in a target sequence (e.g., an amplified target sequence). For example, the calibrator probe and/or the indicator probe can include at least one modified nucleotide that is located 1, 2, 3, 4, 5, 6, 7, 8, 9, or 10 nucleotides downstream or upstream of a mutation in a target sequence. In another example, one or more modified nucleotides can be present in a region in proximity to or within a conserved region when bound to a target sequence and/or a region in proximity to or within a mutated region when bound to a target sequence. In some embodiments, the probe can include a base modification that allows cleavage of the probe (e.g., by an enzyme). In some embodiments, the probe can include a base modification that allows cleavage of the probe and release of a detectable signal. Non-limiting examples of such base modifications include an RNA base in DNA probe for RNaseH2-based cleavage of RNA/DNA hybrids, an abasic nucleotide analogues (e.g., a tetrahydrofuran residue (THF)), and a C—O—C linker for exonuclease or endonuclease cleavage, and a deoxyribose (dR)-group for exonuclease or endonuclease cleavage.

A probe can include one or more detectable labels, e.g., detectable labels known in the art or described herein. The one or more detectable labels can be included anywhere in the probe, e.g., 3′ end of the probe, 5′ end of the probe, internally, or a combination thereof.

### Detectable Labels

The calibrator probe and/or the indicator probe for use in methods described herein can be conjugated to a detectable label. The detectable label conjugated to the calibrator probe can be the same as or different from the detectable label conjugated to the indicator probe.

As used herein, a “detectable label” refers to any molecule that is capable of releasing a detectable signal, either directly or indirectly. In some embodiments, the detectable label can be a fluorophore (e.g., Cy5). As used herein, the term “fluorophore” (also referred to as “fluorescent label” or “fluorescent dye”) refers to moieties that absorb light energy at a defined excitation wavelength and emit light energy at a different wavelength.

Examples of fluorophores include, without limitation, cyanine derivatives (e.g., cyanine, indocarbocyanine, oxacarbocyanine, thiacarbocyanine, and merocyanine), xanthene derivatives (e.g., fluorescein, rhodamine, Oregon green, eosin, and Texas Red®), naphthalene derivatives (e.g., dansyl and prodan derivatives), pyrene derivatives (e.g., cascade blue), oxadiazole derivatives (e.g., pyridyloxazole, nitrobenzoxadiazole, and benzoxadiazole), oxazine derivatives (e.g., Nile red, Nile blue, cresyl violet, and oxazine 70), acridine derivatives (e.g., proflavin, acridine orange, and acridine yellow), arylmethine derivatives (e.g., auramine, crystal violet, and malachite green), tetrapyrrole derivatives (e.g., porphin, phthalocyanine, and bilirubin), coumarin derivatives, or fluorescent proteins (e.g., green fluorescent protein).

In some embodiments, the detectable label is a radioactive label (e.g., ³⁵S, ¹²⁵I, or radioactive phosphates, such as ³²P or ³³P). In some embodiments, the detectable label is a chemiluminescent label (e.g., acridinium esters or ruthenium esters). Detectable labels include other labels such as dyes, colorimetric labels, biotin, avidin, streptavidin, digoxigenin, haptens, quantum dots, nanoparticles, and the like. In some non-limiting examples, two or more different detectable labels may be used for each probe (e.g., a nanoparticle and a label, biotin and a label, etc.).

In some embodiments, the detectable label comprises a fluorophore donor and an acceptor moiety such as a fluorophore acceptor or a fluorescence quencher. Any fluorophore donor and acceptor moiety suitable for fluorescence resonance energy transfer (FRET) can be used in methods disclosed herein. Fluorophore donor and acceptor pairs for use in methods described herein are known in the art and commercially available, e.g., from Life Technologies (Carlsbad, CA), GE Healthcare (Piscataway, NJ), Integrated DNA Technologies (Coralville, IA), and Roche Applied Science (Indianapolis, IN). For example and without limitation, a probe can include both a donor moiety and an acceptor moiety that are each attached to differing positions on the probe.

As used herein, a “fluorophore donor” refers to a fluorophore that, upon absorbing light, can transfer excitation energy to a fluorophore acceptor or a fluorescence quencher. Non-limiting examples of a fluorophore donor include Alexa Fluor® 488, Alexa Fluor® 568, Alexa Fluor® 594, Alexa Fluor® 647, Cy™ 2, Cy™ 3, BODIPY™, GFP, fluorescein, IEDANS, EDANS, or a lanthanide metal (e.g., europium, terbium, and samarium).

As used herein, a “fluorophore acceptor” refers to a fluorophore that can accept excitation energy transferred by a fluorophore donor and use the transferred energy to emit light at its own characteristic emission wavelength spectrum. Non-limiting examples of a fluorophore acceptor include Cy™ 3, Cy™ 5, R-Phycoerythrin (R-PE), allophycocyanin (APC), Alexa Fluor® 532, Alexa Fluor® 546, Alexa Fluor® 610, Alexa Fluor® 647, BODIPY™, fluorescein, and YFP.

As used herein, a “fluorescence quencher” refers to a non-fluorescent molecule that can accept energy from an excited fluorophore, thereby reducing the fluorescence signal of the fluorophore. Non-limiting examples of a fluorescence quencher include Dabcyl, Tamra, Black Hole Quenchers, FAM, HEX, ROX, VIC, Cy™ 5.5, Texas Red®, Iowa Black®, Deep Dark Quenchers, Eclipse®, and QSY®.

It should be understood that a fluorophore can be a fluorophore donor when paired with one fluorophore, and it can be a fluorophore acceptor when paired with another fluorophore. For example, Cy™ 3 is a fluorophore donor when paired with Cy™ 5, and Cy™ 3 is a fluorophore acceptor when paired with Cy™ 2.

### Amplified Target Nucleic Acids

Methods described herein detect a mutation in an amplified target nucleic acid. The term “target nucleic acid” refers to a nucleic acid whose presence or absence in a sample is to be detected. The term “amplified target nucleic acid” refers to an amplification product of the target nucleic acid. Any method known in the art or described herein can be used to amplify a target nucleic acid to produce an amplified target nucleic acid.

An amplified target nucleic acid can include any sequence, e.g., a naturally occurring sequence or an artificially occurring sequence. The target nucleic acid can be obtained from one or more sources, e.g., a virus, a bacteria, a fungus, or a mammal. Accordingly, in some embodiments, the amplified target nucleic acid is a viral nucleic acid, a bacterial nucleic acid, a fungal nucleic acid, or a mammalian nucleic acid.

Any amplified target nucleic acid can be analyzed by methods described herein. In some embodiments, an amplified target nucleic acid comprises a nucleic acid from a drug resistant bacteria, e.g., genomic DNA from a drug resistant bacteria. For example, the amplified target nucleic acid comprises a nucleic acid from Mycobacterium tuberculosis. In some embodiments, the amplified target nucleic acid comprises a rifampicin resistance-determining region (RRDR) in a rpoB gene from Mycobacterium tuberculosis. In some embodiments, the RRDR comprises a mutation in a codon selected from codon 511, codon 516, codon 526, codon 531, codon 533, codon 577, or a combination thereof. See Examples below.

An amplified target nucleic acid can be completely complementary or substantially complementary to all or a portion of a portion of a calibrator probe and/or an indicator probe. For example, the amplified target nucleic acid and the calibrator probe can be at least 75%, at least 80%, at least 85%, at least 90%, at least 95%, at least 96%, at least 97%, at least 98%, at least 99%, or more complementary to each other over any useful region (e.g., over a region of 8, 9, 10, 11, 12, 13, 14, 15, 16, 17, 18, 19, 20, 21, 22, 23, 24, 25, 30, 35, 40, 45, 50, or more nucleotides). Alternatively, or in addition to, the amplified target nucleic acid and the indicator probe can be at least 75%, at least 80%, at least 85%, at least 90%, at least 95%, at least 96%, at least 97%, at least 98%, at least 99%, or more complementary to each other over any useful region (e.g., over a region of 8, 9, 10, 11, 12, 13, 14, 15, 16, 17, 18, 19, 20, 21, 22, 23, 24, 25, 30, 35, 40, 45, 50, or more nucleotides).

An amplified target nucleic acid can comprise DNA, RNA, or a combination of DNA and RNA. The amplified target nucleic acid can include one or more mutations, e.g., 1, 2, 3, 4 or more mutations.

### II. Dual Probe Assays for Detecting a Mutation in an Amplified Target Nucleic Acid

Aspects of the present disclosure provide methods for detecting a mutation in an amplified target nucleic acid using a calibrator probe and an indicator probe. To perform the assay method described herein, a sample comprising an amplified target nucleic acid, a calibrator probe, and an indicator probe is incubated under conditions sufficient for hybridizing the calibrator probe and/or the indicator probe to the amplified target nucleic acid. Hybridization of the calibrator probe and the indicator probe to the amplified target nucleic acid can be detected by detecting a signal released from the detectable label attached to the probe.

Methods described herein encompass incubating a sample for any period of time sufficient for amplification of a target nucleic acid and/or hybridization of the amplified target nucleic acid to a calibrator probe and/or an indictor probe. In some embodiments, the sample is incubated for about 30 minutes to about 120 minutes, e.g., about 45 minutes to about 120 minutes, about 60 minutes to about 120 minutes, about 75 minutes to about 120 minutes, about 90 minutes to about 120 minutes, about 105 minutes to about 120 minutes, about 30 minutes to about 105 minutes, about 30 minutes to about 90 minutes, about 30 minutes to about 90 minutes, about 30 minutes to about 75 minutes, about 30 minutes to about 60 minutes, or about 30 minutes to about 45 minutes.

Methods described herein encompass incubating a sample at any temperature sufficient for amplification of a target nucleic acid and/or hybridization of the amplified target nucleic acid to a calibrator probe and/or an indictor probe. In some embodiments, methods described herein comprise incubating a sample at any temperature sufficient for nucleic acid amplification. In some embodiments, the sample is incubated at a temperature of about 20 to about 75° C., e.g., about 25 to about 75° C., about 30 to about 75° C., about 35 to about 75° C., about 40 to about 75° C., about 45 to about 75° C., about 50 to about 75° C., about 55 to about 75° C., about 60 to about 75° C., about 65 to about 75° C., about 70 to about 75° C., about 20 to about 70° C., about 20 to about 65° C., about 20 to about 60° C., about 20 to about 55° C., about 20 to about 50° C., about 20 to about 45° C., about 20 to about 40° C., about 20 to about 35° C., about 20 to about 30° C., or about 20 to about 25° C.

Any amount of a calibrator probe and an indicator probe suitable for hybridization of the probe to an amplified target nucleic acid can be used in methods described herein. In some embodiments, the sample can comprise a calibrator probe at a concentration of 0.1 to 10 μM, e.g., 0.25 to 10 μM, 0.5 to 10 μM, 1 to 10 μM, 2.5 to 10 μM, 5 to 10 μM, 7.5 to 10 μM, 0.1 to 7.5 μM, 0.1 to 5 μM, 0.1 to 2.5 μM, 0.1 to 1 μM, 0.1 to 0.5 μM, or 0.1 to 0.25 μM. In some embodiments, the sample can comprise an indicator probe at a concentration of 0.1 to 10 μM, e.g., 0.25 to 10 μM, 0.5 to 10 M, 1 to 10 μM, 2.5 to 10 μM, 5 to 10 μM, 7.5 to 10 μM, 0.1 to 7.5 μM, 0.1 to 5 μM, 0.1 to 2.5 μM, 0.1 to 1 μM, 0.1 to 0.5 μM, or 0.1 to 0.25 μM.

Methods described herein encompass determining an absence or a presence of a mutation in a sample based on a score value determined from signals from the indicator probe and the calibrator probe. A deviation of a score value from a control value indicates the absence or the presence of the mutation. The score value and/or the control value can be determined using methods known in the art or described herein.

In some embodiments, the score value can be determined using a statistical model described in FIGS. 3A-3B. The variables used in Equations 1-5 are depicted in the real-time amplification calibration curve shown in FIG. 3A. Equations 1-5 and Z-score are shown in FIG. 3B. In some embodiments, the score value is determined using Equation 1, Equation 2, Equation 3, Equation 4, Equation 5, Z-score, or a combination thereof.

Equations 1-5 are provided below:

$$\begin{matrix}S = {f}_{ie} / {f}_{ce} & Equation 1 \\ S = ( {f}_{ie} - {f}_{ib} ) / ( {f}_{ce} - {f}_{cb} ) & Equation 2 \\ S = ( {f}_{ie} / {f}_{ib} ) / ( {f}_{ce} / {f}_{cb} ) & Equation 3 \\ S = ( {f}_{ie} - {f}_{ib} ) - ( {f}_{ce} - {f}_{cb} ) & Equation 4 \\ S = {f}_{ie} - {f}_{ce} & Equation 5\end{matrix}$$

wherein:
f_{cb}: baseline signal of calibrator probe;
f_{ce}: end point signal or amplification plateau phase signal of calibrator probe;
f_{ib}: baseline signal of indicator probe; and
fᵢₑ: end point signal or amplification plateau phase signal of indicator probe.

In some embodiments, the control score is a score value determined from the signal from the calibrator probe and the signal from the indicator probe in a control sample comprising an amplified target nucleic acid without a mutation. In some embodiments, the signal from the calibrator probe comprises a signal from the first detectable label and the signal from the indicator probe comprises a signal from the second detectable label. In some embodiments, the signal from the calibrator probe comprises a signal from the second detectable label and the signal from the indicator probe comprises a signal from the first detectable label.

Control samples comprising an amplified target nucleic acid without a mutation can be referred to as negative control samples or WT samples. Control samples comprising an amplified target nucleic acid with a mutation can be referred to as positive control samples or mutant samples.

In some embodiments, the control value is based on an absolute value determined from the signal from the calibrator probe and the signal from the indicator probe in a negative control sample comprising the amplified target nucleic acid without the mutation. Alternatively, or in addition to, the control value is based on an absolute value determined from the signal from the first detectable label and the signal from the second detectable label in a positive control sample comprising the amplified target nucleic acid with the mutation.

In some embodiments, the score value comprises a Z-score of

$$Z = \vert \frac{X - μ}{σ} \vert ,$$

X comprises S score (FIG. 3B) from the test sample based on the signal from the calibrator probe and the indicator probe, μ comprises the mean of S score (FIG. 3B) from a plurality of negative control samples comprising a target nucleic acid without a mutation (e.g., an n number of negative control samples, including between 5 to 10 samples), and σ comprises a standard deviation of S score from the plurality of negative control samples comprising the target nucleic acid without the mutation.

In some embodiments, the control value is a predetermined value. The predetermined value can be a single cut-off (threshold) value (T), such as a median or mean, or a score that defines the boundaries of an upper or lower quartile, tertile, or other segment of a score value that is determined to be statistically different from the other segments. The control value can be a range of cut-off (or threshold) values, such as a confidence interval. The control value can be established based upon comparative groups. Alternatively, or in addition to, the control value can be a range.

In some embodiments, the determining the absence or the presence of the mutation comprises assessing a signal from the calibrator probe and the indicator probe, calculating a Z-score, comparing the Z-score to a threshold value of T, and determining the presence of the mutation in the amplified target nucleic acid based on a comparison of the Z-score to T. In such instances, a mutation is present in the amplified target nucleic acid when the Z-score is greater than the threshold value (e.g., Z>T) and/or a mutation is absent in the amplified target nucleic acid when the Z-score is less than or equal to the threshold value (e.g., Z≤T).

Methods described herein can comprise amplifying a target nucleic acid to produce an amplified target nucleic acid. In such instances, the sample (e.g., the test sample and/or the control sample) can comprise one or more additional components for amplifying the target nucleic acid. For example, the sample can comprise 3′-amino-2′,3′-dideoxyribonucleotide 5′-triphosphates (nNTPs), one or more additional primers, a buffer, a DNA polymerase, a reverse transcriptase or a combination thereof.

The target nucleic acid can be amplified using any method known in the art or described herein. In some embodiments, the target nucleic acid is amplified using isothermal amplification. Non-limiting examples of isothermal amplification include nucleic acid sequence based amplification (NASBA), helicase-dependent amplification (HDA), loop-mediated isothermal amplification (LAMP), strand displacement amplification (SDA), and recombinase polymerase amplification (RPA).

Methods described herein encompass detecting any mutation in an amplified target nucleic acid. Non-limiting examples of a mutation include an insertion, a deletion, and a substitution. In some embodiments, the mutation is a single nucleotide polymorphism.

Methods described herein encompass detecting one or more mutations in an amplified target nucleic acid. In such instances, methods described herein comprise use of one or more indicator probes. In some embodiments, an indicator probe is designed to detect more than one mutation, e.g., 2, 3, 4, or more mutations. In some embodiments, each indicator probe is used to detect a different mutation, e.g., an indictor probe for detecting a first mutation and another indicator probe for detecting a second mutation. When using more than one indicator probe in methods described herein, each indicator probe can comprise a different detectable label.

Methods described herein encompass detecting a signal from a detectable label using any method known in the art or described herein. In some embodiments, methods comprise detecting a colorimetric signal, a fluorescent signal, a chemiluminescent signal, or a combination thereof. For example, the amplified target nucleic acid can be fluorescently labeled (e.g., FAM-labeled), the indicator probe and the calibrator probe can be non-fluorescently labeled (e.g., biotin-labeled or digoxigenin-labeled), and detection can be performed using gold nanoparticles (AuNPs) in a lateral flow immunoassay. In some embodiments, the indicator probe and/or the calibrator probe can be attached to different catalytic substrates to yield different chemiluminescence signals in a multiplexed system. In some embodiments, the indicator probe and/or the calibrator probe can be attached to different nanoparticles that produce different detectable signals when the nanoparticles aggregate upon target detection.

In some embodiments, methods described herein comprise detecting a signal from a detectable label using real-time detection and/or end-point detection. In some embodiments, the signal from the detectable label is detected using a fluorescent reader, an optical signal reader, a microfluidic device, or a combination thereof. Examples of suitable microfluidic systems, methods, and devices are described in, e.g., U.S. Pat. Nos. 5,976,336; 7,419,784; 7,276,330; 7,081,190; 5,948,227; 6,042,710; 6,440,284, each of which is incorporated herein by reference in its entirety.

The present disclosure also includes use of methods described herein for a variety of purposes that involve detecting a mutation including, but not limited to, clinical purposes such as identifying a subject having a disease associated with a mutation, selecting a treatment, assessing efficacy of a treatment, monitoring a treatment, and non-clinical purposes such as research purposes.

### III. Kits

The present disclosure also provides kits for detecting a mutation in an amplified target nucleic acid. Such kits include a calibrator probe and an indicator probe. The calibrator probe and/or the indicator probe can be conjugated to a detectable label. The kit can include one or more reagents for amplifying a target nucleic acid to produce an amplified target nucleic acid (e.g., one or more primers, one or more enzymes (e.g., a polymerase), one or more buffers, nNTPs, etc.) and/or instructions for practicing any of the methods described herein. Instructions supplied in the kits of the present disclosure are typically written instructions on a label or a package insert.

### EXAMPLES

In order that the invention described may be more fully understood, the following examples are set forth. The examples described in this application are offered to illustrate the methods and compositions provided herein and are not to be construed in any way as limiting their scope.

### Example 1: Detection of Mutations Linked to Rifampicin Drug Resistance Using Dual Probe Detection

This Example describes detecting mutations that cause rifampicin (RIF) drug resistance using a non-limiting dual probe detection method.

RIF is one of the most common antibacterial agents used in treatment plan for Mycobacterium tuberculosis infections. Mutations in the 81-bp long region of the rpoB gene called the RIF resistance-determining region (RRDR) can lead to resistance to RIF (also referred to as RIF-resistance). Ninety percent of the RIF-resistance is linked to mutations in this key region, with most mutations occurring at codons 516, 526, and 531.

Materials and Methods: LAMP primers were designed to amplify the RRDR region of the rpoB gene. The LAMP primers are designed such that one of the loops of the amplicon contains the mutation-prone region and the other loop contains a conserved sequence of the rpoB gene. In this non-limiting example, the calibrator probe is a molecular beacon probe that is designed to detect the conserved sequence of one of the loops of the amplicon, and the indicator probe is a molecular beacon probe that is designed to target codons 526 and 531 in the mutation-prone region of the other loop in the amplicon (Table 1 and FIG. 5A). Final concentrations of primers and probes in reactions are as follows: 1.6 μM of FIB/BIP, 0.2 μM of F3/B3, 0.3 μM of LB/LF, and 0.4 μM of calibrator and indicator probes. The Bst polymerase used for the LAMP reaction was obtained from Meridian Biosciences. Mycobacterium tuberculosis genomic DNA was obtained from ATCC. Plasmids carrying either the wild type sequence or the mutated target nucleic acid sequence were synthesized by GenScript (Table 2). The CFX96 Touch Real-Time PCR Detection System from Bio-Rad was used for signal readout. Reactions were incubated at 66° C. for 1 hour for the isothermal amplification. Real-time fluorescence data was collected over 1 hour during the amplification step.

TABLE 1
Sequences of primers and probes

| Oligo Name | Sequences (5′-3′) |
|---|---|
| F3 | AGCGGATGACCACCCAG |
| B3 | TGCACGTCGCGGACCT |
| FIP | CTTGATCGCGGCGACCACCG GACGTGGAGGCGATCACA |
| BIP | CAGAACAACCCGCTGTCGGCACGCTCACGTGACAGACC |
| LF | GCCGGATGTTGATCAACG |
| LB | CCACAAGCGCCGACTG |
| Calibrator | FAM-CGCGAGCCGGATGTTGATCAACGTCTGCTCGCG- |
| Probe | BHQ1 |
| Indicator | Cy5-CGCGAGACC[+C][+A][+C]AAGCGCCGACTG |
| Probe | [+T][+C][+G]GCGCTCGCG-BHQ2* |

*: [+] is LNA modified base.

TABLE 2
81-bp RRDR sequences in plasmids

| Plasmid | | |
|---|---|---|
| ID | 81-bp RRDR sequences | Codon |
| Wild | GGCACCAGCCAGCTGAGCCAATTCATGGACC | His-526, |
| | AGAACAACCCGCTGTCGGG | |
| Type | GTTGACCCACAAGCGCCGACTGTCGGCGCTG | Ser-531 |
| | | (Wt) |
| RIF 1 | GGCACCAGCCAGCTGAGCCAATTCATGGACC | Leu-526, |
| | AGAACAACCCGCTGTCGGGGTTGACCCtCAA | Ser-531 |
| | GCGCCGACTGTCGGCGCTG | |
| RIF 2 | GGCACCAGCCAGCTGAGCCAATTCATGGACC | Tyr-526, |
| | AGAACAACCCGCTGTCGGGGTTGACCtACAA | Ser-531 |
| | GCGCCGACTGTCGGCGCTG | |
| RIF 3 | GGCACCAGCCAGCTGAGCCAATTCATGGACC | His-526, |
| | AGAACAACCCGCTGTCGGGGTTGACCCACAA | Trp-531 |
| | GCGCCGACTGTgGGCGCTG | |

Results: The recognition sites for the indicator probe (Probe I) and the calibrator probe (Probe C) are shown in FIG. 5A. The calibrator probe (Probe C) recognizes the conserved sequence in the single stranded region in one of the loops of the dumbbell-shaped amplicon produced during the isothermal amplification. The indicator probe (Probe I) recognizes the mutation hotspot region (containing codon 526 and codon 531) in the single stranded region in the second loop of the dumbbell-shaped amplicon produced during the isothermal amplification. Schematic representations of possible results are shown in FIG. 2.

Fluorescence signals were obtained using Mycobacterium tuberculosis genomic DNA is (ATCC-25177D) and the mean (μ) and the standard deviation (σ) of S_{wt} were calculated (Table 3). The standard deviation (σ) of S_{wt} was calculated using Equation 3.

Fluorescence signals were obtained using the calibrator probe (Probe C) and the indicator probe (Probe I) and different plasmid types (FIGS. 5B-5E). As shown in FIG. 5B, when the sample includes the wild type plasmid, the indicator probe (solid line) produced higher signals compared to the calibrator probe, with a Z-score (Z) of 1.62. As shown in FIGS. 5C-5E, when the sample includes the mutant plasmid RIF1 (FIG. 5C), RIF2 (FIG. 5D), or RIF3 (FIG. 5E), the signals from the indicator probe were dramatically decreased. After calibration by the calibrator probe, the Z-score (Z) of RIF1, RIF2, and RIF3 are 10.60, 9.28, and 6.65, respectively.

Total 47 plasmid samples with different concentration were tested in validation study. Using the decision chart shown in FIG. 4, 7 out of 47 samples with f_{ce}/f_{cb}<1.1 were determined to be invalid. The raw signals, calibrated signal, and Z-score of all 47 samples are shown in Table 4. Z-score distributions for 11 wild type plasmid samples and 29 RIF mutant plasmid samples are shown in FIG. 6A. The higher Z score indicates a larger deviation of calibrated signals compared to the training dataset. FIG. 6B shows that 100% accuracy for classifying samples as wild type or mutants can be achieved when the Z-score threshold is set to 5.

Taken together, the results described herein demonstrate that dual probe detection allows accurate detection of mutations such as RIF-resistance linked mutations in the RRDR of the rpoB gene.

TABLE 3
S_{wt} calculation using Mycobacterium tuberculosis genomic DNA

| Standards | f_{ib} | fᵢₑ | f_{cb} | f_{ce} | *S_{wt} = (fᵢₑ/f_{ib})/(f_{ce}/f_{cb}) |
|---|---|---|---|---|---|
| MTB-gDNA | 2664 | 3738 | 2891 | 3368 | 1.20 |
| MTB-gDNA | 2668 | 4044 | 2903 | 3438 | 1.28 |
| MTB-gDNA | 2657 | 3773 | 2940 | 3447 | 1.21 |
| MTB-gDNA | 2662 | 3933 | 2842 | 3341 | 1.26 |
| MTB-gDNA | 2675 | 3944 | 2822 | 3314 | 1.26 |
| MTB-gDNA | 2650 | 3794 | 2910 | 3400 | 1.23 |
| MTB-gDNA | 2649 | 3941 | 2785 | 3290 | 1.26 |
| MTB-gDNA | 2650 | 4020 | 2796 | 3337 | 1.27 |
| MTB-gDNA | 2632 | 3807 | 2761 | 3264 | 1.22 |
| MTB-gDNA | 2639 | 3843 | 2762 | 3224 | 1.25 |
| MTB-gDNA | 2633 | 3758 | 2746 | 3208 | 1.22 |
| MTB-gDNA | 2634 | 3741 | 2779 | 3301 | 1.20 |

*Note:
Mean (μ) S_{wt} = 1.24, Standard Deviation (σ) S_{wt} = 0.03

TABLE 4
Z-score for the 47 samples tested using the dual-probe method

| | Concentration | | | | | | |
|---|---|---|---|---|---|---|---|
| Plasmid-ID | (cps/reaction) | f_{ib} | fᵢₑ | f_{cb} | f_{ce} | Sₛ = (fᵢₑ/f_{ib})/(f_{ce}/f_{cb}) | Z-Score |
| Wild Type | 4000 | 2615 | 3864 | 2705 | 3138 | 1.27 | 1.26 |
| Wild Type | 4000 | 2608 | 3927 | 2702 | 3173 | 1.28 | 1.62 |
| Wild Type | 4000 | 2591 | 3697 | 2727 | 3169 | 1.23 | 0.55 |
| Wild Type | 1332 | 2585 | 3717 | 2695 | 3139 | 1.23 | 0.27 |
| Wild Type | 1332 | 2572 | 3839 | 2646 | 3168 | 1.25 | 0.20 |
| Wild Type | 1332 | 2562 | 3608 | 2608 | 3093 | 1.19 | 2.13 |
| Wild Type | 444 | 2572 | 3500 | 2621 | 3138 | 1.14 | 4.15 |
| Wild Type | 444 | 2580 | 3753 | 2696 | 3123 | 1.26 | 0.57 |
| Wild Type | 444 | 2583 | 3713 | 2642 | 3112 | 1.22 | 0.83 |
| Wild Type | 148 | 2589 | 3668 | 2684 | 3135 | 1.21 | 1.13 |
| Wild Type | 148 | 2553 | 3370 | 2587 | 3015 | 1.13 | 4.31 |
| RIF 1 | 4000 | 2593 | 2965 | 2612 | 3033 | 0.98 | 10.18 |
| RIF 1 | 4000 | 2590 | 2941 | 2621 | 3055 | 0.97 | 10.60 |
| RIF 1 | 4000 | 2594 | 3268 | 2648 | 3105 | 1.07 | 6.62 |
| RIF 1 | 1332 | 2580 | 2994 | 2628 | 3071 | 0.99 | 9.85 |
| RIF 1 | 1332 | 2583 | 3037 | 2629 | 3065 | 1.01 | 9.24 |
| RIF 1 | 1332 | 2595 | 2857 | 2660 | 3129 | 0.94 | 12.11 |
| RIF 1 | 444 | 2586 | 2998 | 2632 | 3083 | 0.99 | 9.99 |
| RIF 1 | 444 | 2582 | 3077 | 2637 | 3127 | 1.00 | 9.38 |
| RIF 1 | 444 | 2588 | 2839 | 2642 | 3101 | 0.93 | 12.15 |
| RIF 1 | 148 | 2578 | 3008 | 2637 | 3037 | 1.01 | 9.05 |
| RIF 1 | 148 | 2575 | 2724 | 2617 | 2658 | — | Invalid * |
| RIF 1 | 148 | 2581 | 2580 | 2636 | 2647 | — | Invalid * |
| RIF 2 | 4000 | 2590 | 3047 | 2608 | 3048 | 1.01 | 9.31 |
| RIF 2 | 4000 | 2589 | 3070 | 2646 | 3112 | 1.01 | 9.23 |
| RIF 2 | 4000 | 2649 | 3108 | 2988 | 3147 | — | Invalid * |
| RIF 2 | 1332 | 2588 | 3090 | 2642 | 3132 | 1.01 | 9.28 |
| RIF 2 | 1332 | 2591 | 3038 | 2691 | 3132 | 1.01 | 9.28 |
| RIF 2 | 1332 | 2586 | 3118 | 2640 | 3125 | 1.02 | 8.84 |
| RIF 2 | 444 | 2594 | 3047 | 2666 | 3143 | 1.00 | 9.72 |
| RIF 2 | 444 | 2578 | 2897 | 2647 | 3129 | 0.95 | 11.52 |
| RIF 2 | 444 | 2584 | 2949 | 2655 | 3127 | 0.97 | 10.81 |
| RIF 2 | 148 | 2590 | 2591 | 2773 | 2796 | — | Invalid * |
| RIF 2 | 148 | 2571 | 3165 | 2626 | 3093 | 1.05 | 7.78 |
| RIF 2 | 148 | 2582 | 3118 | 2854 | 3196 | 1.08 | 6.48 |
| RIF 3 | 4000 | 2601 | 3280 | 2622 | 3058 | 1.08 | 6.35 |
| RIF 3 | 4000 | 2599 | 3287 | 2647 | 3098 | 1.08 | 6.38 |
| RIF 3 | 4000 | 2585 | 3264 | 2636 | 3096 | 1.08 | 6.59 |
| RIF 3 | 1332 | 2582 | 3240 | 2629 | 3066 | 1.08 | 6.56 |
| RIF 3 | 1332 | 2585 | 3266 | 2629 | 3114 | 1.07 | 6.94 |
| RIF 3 | 1332 | 2587 | 3290 | 2644 | 3128 | 1.07 | 6.60 |
| RIF 3 | 444 | 2587 | 3320 | 2644 | 3137 | 1.08 | 6.34 |
| RIF 3 | 444 | 2577 | 3266 | 2633 | 3101 | 1.08 | 6.57 |
| RIF 3 | 444 | 2590 | 3317 | 2663 | 3141 | 1.09 | 6.17 |
| RIF 3 | 148 | 2578 | 2580 | 2634 | 2663 | — | Invalid * |
| RIF 3 | 148 | 2576 | 2577 | 2633 | 2664 | — | Invalid * |
| RIF 3 | 148 | 2573 | 2573 | 2645 | 2667 | — | Invalid * |

* These samples are determined Invalid based on f_{ce}/f_{cb} < 1.1 (decision chart of FIG. 4).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for detecting a mutation in an amplified target nucleic acid in a sample, the method comprising:
incubating a test sample comprising:
an amplified target nucleic acid;
a calibrator probe comprising a first nucleic acid sequence that is sufficiently complementary to a second nucleic acid sequence of the amplified target nucleic acid, wherein the second nucleic acid sequence of the amplified target nucleic acid comprises a wild-type sequence, and wherein the calibrator probe comprises a first detectable label;
an indicator probe comprising a third nucleic acid sequence that is sufficiently complementary to a fourth nucleic acid sequence of the amplified target nucleic acid, wherein the fourth nucleic acid sequence of the amplified target nucleic acid comprises an absence or a presence of a mutation, and wherein the indicator probe comprises a second detectable label;
wherein the test sample is incubated under conditions sufficient for hybridizing the calibrator probe and/or the indicator probe to the amplified target nucleic acid;
detecting a signal from the first detectable label and a signal from the second detectable label; and
determining the absence or the presence of the mutation in the fourth nucleic acid sequence of the amplified target nucleic acid based on a score value determined from the signal from the first detectable label and the signal from second detectable label,
wherein a deviation of the score value from a control value indicates the presence of the mutation in the fourth nucleic acid sequence of the amplified target nucleic acid in the test sample.

2. The method of claim 1, wherein the score value comprises a Z-score of
$$Z = \vert \frac{X - μ}{σ} \vert ,$$
X comprises a signal from the test sample based on the signal from the first detectable label and the signal from second detectable label, μ comprises a signal from a plurality of negative control samples comprising an amplified target nucleic acid without a mutation, and σ comprises a standard deviation of the signal from the plurality of negative control samples comprising the amplified target nucleic acid without the mutation.

3. The method of claim 1, wherein the control value is based on an absolute value determined from the signal from the first detectable label and the signal from the second detectable label in a negative control sample comprising the amplified target nucleic acid without the mutation and/or based on an absolute value determined from the signal from the first detectable label and the signal from the second detectable label in a positive control sample comprising the amplified target nucleic acid with the mutation.

4. The method of claim 1, wherein the control value is a predetermined threshold value.

5. The method of claim 1, wherein said determining the absence or the presence of the mutation comprises:
assessing a presence or an absence of a calibrator negative signal or an indicator negative signal;
calculating a Z-score of
$$Z = \vert \frac{X - μ}{σ} \vert ,$$
wherein X comprises a signal from the test sample based on the signal from the first detectable label and the signal from second detectable label, μ comprises a signal from a plurality of negative control samples comprising an amplified target nucleic acid without a mutation, and σ comprises a standard deviation of the signal from the plurality of negative control samples comprising the amplified target nucleic acid without the mutation;
comparing the Z-score to a threshold value of T; and
determining the presence of the mutation in the fourth nucleic acid sequence of the amplified target nucleic acid when the Z-score is greater than the threshold value (e.g., Z>T) or determining the absence of the mutation in the fourth nucleic acid sequence of the amplified target nucleic acid when the Z-score is less than or equal to the threshold value (e.g., Z≤T).

6. The method of claim 1, wherein the mutation is an insertion, a deletion, a substitution, or a single nucleotide polymorphism.

7. (canceled)

8. (canceled)

9. The method of claim 1, wherein the amplified target nucleic acid is a viral nucleic acid, a bacterial nucleic acid, a fungal nucleic acid or a mammalian nucleic acid; or wherein the amplified target nucleic acid comprises genomic DNA.

10. The method of claim 1, wherein the amplified target nucleic acid is a nucleic acid from Mycobacterium tuberculosis or a rifampicin resistance-determining region (RRDR) from Mycobacterium tuberculosis.

11. (canceled)

12. The method of claim 10, wherein the RRDR comprises a mutation in a codon selected from codon 511, codon 516, codon 526, codon 531, codon 533, codon 577, or a combination thereof.

13. (canceled)

14. The method of claim 1, wherein the calibrator probe is completely complementary to the second nucleic acid sequence of the amplified target nucleic acid and/or the indicator probe is completely complementary to the fourth nucleic acid sequence of the amplified target nucleic acid: or wherein the calibrator probe has a length of 20 to 50 nucleic acids and/or the indicator probe has a length of 20 to 50 nucleic acids.

15. The method of claim 1, wherein the calibrator probe comprises at least one modification and/or wherein the indicator probe comprises at least one modification; and optionally wherein the at least one modification is internally located in the calibrator probe and/or the indicator probe.

16. (canceled)

17. The method of claim 15, wherein the at least one modification of the indicator probe is located within 5 to 10 nucleotides of the mutation in the fourth nucleic acid sequence of the amplified target nucleic acid when the indicator probe is hybridized to the fourth nucleic acid sequence; and/or wherein the at least one modification comprises locked nucleic acid (LNA), a peptide nucleic acid (PNA), a bridged nucleic acid (BNA), an unlocked nucleic acid (UNA), and/or a self-avoiding molecular recognition system (SAMRS).

18. (canceled)

19. The method of claim 1, wherein the first and second detectable labels are selected from the group consisting of a fluorescent label, a radioactive label, a colorimetric, a chemiluminescent label, and/or a dye.

20. The method of claim 19, wherein the first detectable label comprises a first fluorophore and a first quencher, and wherein the second detectable label comprises a second fluorophore and a second quencher.

21. (canceled)

22. (canceled)

23. (canceled)

24. The method of claim 1, wherein the calibrator probe comprises CGCGAGCCGGATGTTGATCAACGTCTGCTCGCG (SEQ ID NO:1) or X-CGCGAGCCGGATGTTGATCAACGTCTGCTCGCG-Y (SEO ID NO:1), and wherein at least one X and Y is a fluorophore and at least one of X and Y is a quencher.

25. (canceled)

26. The method of claim 1, wherein the indicator probe comprises CGCGAGACCCACAAGCGCCGACTGTCGGCGCTCGCG (SEQ ID NO:2) or X-CGCGAGACCCACAAGCGCCGACTGTCGGCGCTCGCG-Y (SEO ID NO:2), and wherein at least one X and Y is a fluorophore and at least one of X and Y is a quencher.

27. (canceled)

28. The method of claim 26, wherein the indicator probe comprises at least one LNA modification at a position selected from positions 10, 11, 12, 25, 26, and 27 in SEQ ID NO:2 or wherein the indicator probe comprises LNA modifications at positions 10, 11, 12, 25, 26, and 27 in SEO ID NO:2.

29. (canceled)

30. The method of claim 1, wherein the calibrator probe and the indicator probe is a molecular beacon.

31. (canceled)

32. The method of claim 1, wherein the sample further comprises 3′-amino-2′,3′-dideoxyribonucleotide 5′-triphosphates (nNTPs), one or more additional primers, a buffer, and/or a DNA polymerase, and optionally wherein the one or more additional primers comprises another indicator probe comprising a fifth nucleic acid sequence that is complementary to a sixth nucleic acid sequence of the amplified target nucleic acid, wherein the sixth nucleic acid sequence of the amplified target nucleic acid comprises an absence or a presence of a mutation, and wherein the another indicator probe comprises a third detectable label.

33. The method of claim 1, further comprising: amplifying the amplified target nucleic acid using an isothermal amplification method, and optionally wherein the isothermal amplification method is selected from nucleic acid sequence based amplification (NASBA), helicase-dependent amplification (HDA), loop-mediated isothermal amplification (LAMP), strand displacement amplification (SDA) or recombinase polymerase amplification (RPA).

34. (canceled)

35. The method of claim 1, wherein detecting the signal from the first, the second, detectable label comprises real-time detection or end point detection and/or wherein detecting the signal from the first, the second detectable label comprises detection using a microfluidic device.

36. (canceled)

37. A kit comprising:
a calibrator probe comprising a first nucleic acid sequence that is sufficiently complementary to a second nucleic acid sequence of an amplified target nucleic acid, wherein the second nucleic acid sequence of the amplified target nucleic acid comprises a wild-type sequence, and wherein the calibrator probe comprises a first detectable label;
an indicator probe comprising a third nucleic acid sequence that is sufficiently complementary to a fourth nucleic acid sequence of the amplified target nucleic acid, wherein the fourth nucleic acid sequence of the amplified target nucleic acid comprises an absence or a presence of a mutation, and wherein the indicator probe comprises a second detectable label;
optionally one or more of the following: 3′-amino-2′,3′-dideoxyribonucleotide 5′-triphosphates (nNTPs), one or more additional primers, a buffer, a DNA polymerase, and/or a reverse transcriptase; and
instructions for performing a method of claim 1.

38. (canceled)


SEQUENCE LISTING (2 sequences)

SEQ ID NO: 1 - DNA, 33 bp, synthetic construct
  source 1..33: mol_type = other DNA
cgcgagccggatgttgatcaacgtctgctcgcg

SEQ ID NO: 2 - DNA, 36 bp, synthetic construct
  source 1..36: mol_type = other DNA
cgcgagacccacaagcgccgactgtcggcgctcgcg